(19) United States
(12) Patent Application Publication
(10) Pub. No.: US 2026/0013784 A1
(43) Pub. Date: Jan. 15, 2026

(54) MULTI-MODAL IMAGING SYSTEM AND METHOD FOR NON-INVASIVE EXAMINATION OF AN OBJECT TO BE EXAMINED

(71) Applicant: JenLab GmbH, Berlin (DE)

(72) Inventors: Karsten KÖNIG, Berlin (DE); Martin WEINIGEL, Jena (DE)

(73) Assignee: JenLab GmbH, Berlin (DE)

(21) Appl. No.: 19/272,841

(22) Filed: Jul. 17, 2025

Related U.S. Application Data
(63) Continuation of application No. 17/040,370, filed on Mar. 12, 2021, now abandoned.

(30) Foreign Application Priority Data
Mar. 23, 2018 (EP) 18163646.5

(51) Int. Cl. A61B 5/00; G02B 21/00; G02B 21/24

(52) U.S. Cl. CPC A61B 5/444; A61B 5/0037; A61B 5/0066; A61B 5/0071; A61B 5/0077; A61B 5/6843; G02B 21/0028; G02B 21/0032; G02B 21/006; G02B 21/0076; G02B 21/244; G02B 21/245; A61B 2562/0247

(57) ABSTRACT

The invention relates to a multi-modal imaging system (2) for non-invasive examination of an examination object (10), comprising a multi-photon imaging system for providing high-resolution detailed images of the examination object (10), which imaging system comprises a radiation source (12), the latter generating an excitation beam (21) of near infrared femtosecond laser radiation for triggering secondary radiation emitted by the examination object (10), and a focusing optical unit (30), by means of which the radiation of the radiation source (12) is directable at a measurement position of the examination object (10), wherein the focusing optical unit (30) and a laser head (14) of the radiation source (12) are provided in a measuring head (4), which is pivotable, rotatable and flexibly positionable freely in space such that the examination of the examination object (10) is performable under any desired solid angle, and comprising at least one confocal detection device, which is at least partly integrated in the measuring head (4) as well and which is configured to receive a signal of the excitation beam (21) of near infrared femtosecond laser radiation, which was diffusely reflected by the examination object (10).

Moreover, a method is specified for non-invasive examination of an examination object (10) using a multi-modal imaging system (2), as is the use of the multi-modal imaging system (2) for examining living matter of the examination object (10)

### PRIORITY DATA

This application is a continuation of U.S. patent application Ser. No. 17/040,370, filed Sep. 22, 2020, which is a 371 nationalization of PCT Application No. PCT/EP2019/057182, filed Mar. 22, 2019, which claims the benefit of European Patent Application Serial No. 18163646.5, filed on Mar. 23, 2018, each of which are incorporated herein by reference.

The invention relates to a multi-modal imaging system and to a method for non-invasive examination of an examination object.

### PRIOR ART

U.S. Pat. No. 5,034,613 A, DE 20 117 294 U1, DE 20 306 122 U1, and DE 10 2010 047 578 A1 disclose rigid and flexible systems for multi-photon imaging using pulsed laser radiation in the near infrared range. By way of example, high-resolution microscopic image fields with edge lengths in the region significantly below 1 mm are generated using multi-photon imaging.

DE 10 2009 029 831 A1 proposes the use of so-called flying optics, wherein an optical unit for focusing excitation radiation and for exciting secondary radiation is not arranged in stationary fashion but moved overall in order to excite, successively in time, different locations of an object to be examined. Here, the spatial excitation is implemented not by beam deflection using rotation and tilt mirrors but by a one-dimensional or multi-dimensional movement of the optical unit as a whole. A disadvantage of this lies in the slow reaction time of the motor-driven optical unit and, as a consequence thereof, a low image recording rate.

DE 10 2011 115 944 A1 has disclosed a flexible nonlinear laser scanning microscope for the non-invasive three-dimensional examination of an examination object, which laser scanning microscope comprises a radiation source which generates an excitation beam for triggering secondary radiation emitted by atoms and molecules and furthermore comprises a flexible transmission optical unit for transmitting the radiation to a measuring head with a focusing optical unit, by means of which the radiation is focused into the test object. Here, the measuring head is connected to the radiation source in a manner such that it is pivotable, rotatable and flexibly positionable freely in space so that microscopy can be carried out under any solid angle.

To facilitate multivalent examination on the human body by the flexibly positionable measuring head, DE 10 2011 115 944 A1 provides for the measuring head to be mechanically supported by a flexible support arm, which is lockable in any position, on a mobile base device which carries along the transmission optical unit. The radiation source for generating pulsed excitation radiation and optical units for aligning the excitation radiation on at least one transmission optical unit to the measuring head are provided in the base device. To achieve the desired accuracy in the flexible beam transmission by the mirror articulated arm, a direction stabilization for the laser radiation is provided, the latter comprising a detector, a control unit and a motor-driven tilt mirror, wherein a correction of the beam direction of the laser beam is implemented by means of the control unit on the basis of a detected deviation in a conjugate target plane within the measuring head. In DE 10 2011 115 944 A1, the superposition of the microscopic image field of the focusing optical unit and the measuring position is afflicted by uncertainty. Since the focusing optical unit has a diameter of approximately 30 mm and a working distance of approximately 0.2 mm from the examination object, the microscopic image field is completely covered by the focusing optical unit.

Moreover, the relative position between the focusing optical unit and the examination object is dependent on the reproducibility of the mechanical coupling between the measuring head and the examination object or an adapter plate for contacting the examination object.

By contrast, the object of the present invention is that of assisting the user with the positioning of the measuring head in relation to the examination object to be examined. The precision of the positioning with which a desired target position (ROI, region of interest) of the examination object to be examined can be represented in high-resolution imaging fashion depends, in particular, on the precision of the mechanical positionability of the focusing optical unit of the multi-modal imaging system.

### DISCLOSURE OF THE INVENTION

In a multi-modal imaging system according to the invention for non-invasive examination of an examination object, comprising an imaging system, in particular a multi-photon imaging system, for providing high-resolution detailed images of the examination object, which imaging system comprises a radiation source, the latter generating an excitation beam of near infrared femtosecond laser radiation for triggering secondary radiation emitted by the examination object, and a focusing optical unit, by means of which the radiation of the radiation source is directable at a measurement position of the examination object, wherein the focusing optical unit and a laser head of the radiation source are provided in a measuring head, which is pivotable, rotatable and flexibly positionable freely in space such that the examination of the examination object is performable under any desired solid angle, provision is made for at least one confocal detection device, which is at least partly integrated in the measuring head as well and which is configured to receive a signal of the excitation beam of near infrared femtosecond laser radiation, which was diffusely reflected by the examination object.

Below, “multi-modal” is understood to mean that two, three or four systems are provided, which are at least partly integrated in one another. The systems can share individual components with one another, for example if one or more measuring or imaging systems use the same radiation source, transmission optical unit, focusing optical unit or the same detector system. Advantageously, a compact arrangement is proposed, in which individual components can be used simultaneously or successively by a plurality of systems. By way of example, the systems can facilitate different imaging modes in succession or at the same time.

In particular, at least the focusing optical unit, by means of which the near infrared femtosecond laser radiation is directable at the examination object, and the confocal detection device are provided integrated in the measuring head and are consequently pivotable, rotatable and flexibly positionable freely in space. Moreover, provision is made for the laser head of the radiation source, too, to be also provided integrated in the measuring head. By way of example, apart from the power supply and the driver, the source of the excitation beam can be completely integrated in the measuring head to this end. Advantageously, the laser beams consequently require no input or output coupling into an articulated arm of the multi-modal imaging system, and so the positioning of the measuring head has no influence on the transmission quality, for example, as a result of which the susceptibility of the system to errors reduces.

Moreover, the measuring head also comprises a minimum of the transmission optical unit, which, in the simplest case, may comprise a scanning unit, a scanning lens, a tube lens, a dichroic beam splitter and the focusing optical unit, for example. By way of example, the resultant image field of such a laser scanning system can be 350 μm×350 μm. Moreover, the measuring head can also comprise an xy-translation stage with a positioning range of, e.g., 5 mm×5 mm. Advantageously, a very compact system is proposed with the integration of various components into the measuring head, said system providing high-resolution detailed recordings of biological and non-biological tissue. Overall, the system is smaller, lighter and more ergonomic than previous systems.

Below, “non-invasive” is understood to mean that the imaging systems do not mechanically penetrate into the examination object. For imaging purposes, non-invasive methods are typically preferred over invasive methods in order to avoid the risks of possible contamination or destruction of the examination object. The proposed multi-modal imaging system for non-invasive examination allows examinations to be carried out on, e.g., living examination objects, for example plants, animals and humans, in particular skin or eye examinations on humans, for example. In particular, depth information from epidermal and/or dermal skin layers, too, can be provided non-invasively with a microscopic resolution, for example to detect skin cancer.

Here, “pivotable, rotatable and flexibly positionable freely in space” is understood to mean that the measuring head is provided for example in mechanically supported fashion on a base device by means of a flexible support arm that is lockable in any desired position. Here, the measuring head and the base device each carry along parts of the multi-modal imaging system. The base device itself can be mobile. By way of example, a part of the radiation source, specifically, e.g., the power supply of the radiation source, can be provided in the base device, as can also computer systems, user interfaces and parts of detector systems for measuring the emitted secondary radiation or the further system for providing overview images of the examination object.

Here, “high-resolution detailed images” is understood to mean that the presented multi-modal imaging system facilitates optical microscopy and tomography with horizontal resolutions in the submicrometer range and scanning regions of several 100 micrometers in three dimensions. By way of example, the image field size of the detailed image is 350 μm×350 μm horizontally and 200 μm vertically.

Below, the “measurement position” of the examination object is also referred to as ROI.

In particular, the multi-photon imaging system can be a multi-photon fluorescence microscope, also a multi-photon fluorescence microscope. A multi-photon fluorescence microscope is a specific light microscope from the group of laser scanning microscopes. In particular, the emitted secondary radiation can be fluorescence radiation, which arises when the examination object absorbs photons and subsequently emits another photon again. The absorbed photon lifts an electron of an atom or molecule of the examination object to a higher energy level and temporarily stores the energy of the exciting photon. After a time characteristic of the corresponding atom or molecule, the electron falls back down to its lower energy level and emits a new photon in the process. While only one photon is required in conventional one-photon fluorescence microscopy for the purposes of exciting the electron to the higher energy level, a plurality of photons are required in multi-photon fluorescence microscopy. Here, the most common method is two-photon fluorescence microscopy (TPFM), wherein the excitation of the electron is implemented by exactly two photons which, in total, have the same energy as required by the electron to be lifted to the higher level. The simultaneous arrival of the two photons is a precondition for the excitation since there typically is no stable intermediate energy level of the electron to be excited. For further information, reference is made to the article by Denk et al., “Two-Photon Laser Scanning Fluorescence Microscopy”, Science, vol. 248, pp. 73-76.

Apart from TPF (two-photon fluorescence), the multi-photon imaging system can also measure second harmonic generation (SHG) signals and third harmonic generation (THG) signals of the excitation radiation. A detector system of the multi-photon imaging system can therefore advantageously comprise one or more detectors for TPF, SHG and THG imaging such that a flexible diagnostic system is available for fluorescent and non-fluorescent substances, particularly in living matter. In particular, simultaneous imaging from TPF, SHG and THG signals from the same location is also possible.

As an alternative or in addition thereto, the detector system of the multi-photon imaging system can comprise an apparatus for time-correlated single photon counting (TCSPC), in particular for capturing the characteristic two-photon fluorescence lifetimes of different endogenous and exogenous fluorophores.

In the present disclosure, “femtosecond laser radiation” denotes, in particular, pulsed laser radiation with pulse durations of, e.g., a few hundred femtoseconds or, preferably, of less than 100 fs, as a distinction from picosecond and attosecond pulses. The term femtosecond laser radiation is also distinguished from CW (continuous wave) lasers. In order to generate high photon densities in the measurement position of the examination object, the excitation beam comprises ultrashort pulsed laser radiation with pulse durations in the femtosecond range.

By way of example, a laser with mode coupling can be considered as a laser. In particular, the laser can use laser diodes as a pump source. By way of example, an erbium fiber laser can be considered as a laser unit, said erbium fiber laser generating an excitation beam with a wavelength of, e.g., 1560 nm, with the excitation radiation being halved to 780 nm accordingly by a frequency doubler. The frequency-doubled radiation is used to excite the atoms and molecules of the examination object.

Alternatively, semiconductor lasers can be provided. Although the repetition rate of the mode-coupled lasers is still slightly too high for time-resolved fluorescence laser microscopy, such a laser can however already be considered for simple intensity measurements.

A further example of a laser emitting suitable femtosecond laser radiation is a titanium sapphire laser with a laser pulse width of less than 200 fs or less than 100 fs, a repetition frequency of 80 MHz or 90 MHz, a power of 0-1.5 W and a wavelength range of 710-920 nm.

By way of example, the focusing optical unit of the multi-photon imaging system comprises a lens system, by means of which the excitation beam is focusable and defocusable on the examination object.

By way of example, the focusing optical unit has a numerical aperture of 0.8 to 1.4, preferably of 1 to 1.4, in particular of, e.g., 1.3, which has been found to be advantageous for multi-photon imaging, in particular for two-photon imaging.

The aforementioned confocal detection device is advantageously at least partly integrated in the measuring head as well, preferably completely integrated therein, and is configured to receive a signal of the excitation beam of near infrared femtosecond laser radiation that was diffusely reflected, e.g., reflected, from the focus volume within the examination object. The diffusely reflected signal can be used for imaging, in particular.

On account of the strong instantaneous reflection signals, significantly shorter pixel dwell times are advantageously required in comparison with autofluorescence-based multi-photon imaging in order to facilitate sufficient morphological imaging of the examination object. This property of the confocal detection device can advantageously be used for “fast” overview imaging of the examination object. Here, overview images can be generated by the checkerboard-like stitching of a multiplicity of adjacent microscopic image fields, for example a multiplicity of adjacent image fields of, e.g., 350×350 μm2. The images are recorded in succession and stitched together in checkerboard-like fashion to form an overview image. To this end, the confocal detection device preferably uses a high numerical aperture of the focusing optical unit, e.g. from 0.8 to 1.4, preferably from 1 to 1.3. The image field sizes of the multi-photon imaging and of the confocal detection device are virtually identical.

Hence, for spatial orientation, i.e., for assisting the user when positioning the measuring head in relation to the examination object to be examined, the use of a complementary imaging method and system with a “large” overview image field stitched together from a multiplicity of smaller image fields is advantageously proposed, the coordinate system of said overview image field permitting, for example, a spatial correlation between the multi-photon imaging and the desired measurement position of the examination object. While conventional stationary multi-photon microscopes offer a restricted area of application on account of their rigid design, the presented multi-modal imaging system is moreover suitable for the multi-valent examination of the human body.

According to a preferred embodiment, at least parts of the detector system of the imaging system are also provided integrated in the measuring head.

The parts of the detector system provided in an integrated fashion are, for example, photomultiplier tubes (PMTs), CCD fields, CMOS fields or similar detectors, which are able to convert the received emitted secondary radiation of the examination object into electronic data signals. By way of the integration of parts of the detector system into the measuring head, a very compact system is advantageously proposed, the latter providing the recording of large-area overview image fields and high-resolution detailed recordings of biological and non-biological tissue.

According to an advantageous embodiment, the multi-modal imaging system comprises at least one further system for providing overview images of the examination object, wherein the further system is at least partly integrated in the measuring head as well and wherein the further system comprises a CCD camera or CMOS camera and/or an optical coherence tomography device. Advantageously, the further system is therefore likewise pivotable, rotatable and flexibly positionable freely in space.

Here, “overview images” are understood to mean images with an edge length of several millimeters, for example. The detection region of the further system for recording overview images can therefore be embodied with the size of, e.g., at least one square millimeter, for example at least 2×2 mm², 5×5 mm² or 10×10 mm².

Below, the term “overview image” is understood to mean both two-dimensional and three-dimensional overview images. By way of example, a three-dimensional image with a large image field is possible in the case of optical coherence tomography.

According to one embodiment, the further system for providing overview images of the examination object is camera-based and comprises a CCD camera or CMOS camera.

As an alternative or in addition thereto, the further system can comprise an optical coherence tomography device. Optical coherence tomography (OCT) advantageously facilitates 2D or 3D overview images, including topographic information of the examination object with a high diagnostic potential. However, the optical coherence tomography device can also be operated in one-dimensional fashion.

Coupling OCT-based overview recordings with the high-resolution multi-photon imaging is made more difficult by different requirements in respect of the respective focusing optical unit because a high numerical aperture of the focusing optical unit is typically required for multi-photon imaging whereas only a small numerical aperture is advantageous for OCT. In respect of these divergent requirements, the present invention offers a solution in which two different optical systems are used. Initially, there is OCT-based overview imaging, followed by high-resolution multi-photon imaging at the corresponding target position (ROI). Both the system for OCT imaging and the system for multi-photon imaging are integrated in a common measuring head and are applied in succession, without mechanical decoupling of the examination object from the measuring head. It is obvious that the target position (ROI) is completely covered by the focusing optical unit in the case of a multi-photon focusing optical unit with a diameter of, e.g., 30 mm and a working distance of approximately 0.2 mm and the latter offers no optical accessibility for the large-area OCT imaging. Therefore, a relatively large distance of, e.g., 10 mm between the multi-photon focusing optical unit and the examination unit is initially set in the Z-direction by way of a linear actuator. A lateral arrangement of the OCT imaging system, tilted at, e.g., 45°, offers the necessary optical accessibility to the target position for the OCT-based overview imaging, without the image field of the OCT imaging system being clipped by the external dimensions of the multi-photon focusing optical unit. The mechanical coupling of the two imaging systems and the reference within the common coordinate system allows the spatial correlation between the two imaging modalities. Here, the OCT imaging system can be realized by a scanning element, for example an xy-galvanometer scanner, an optical system with a further focusing optical unit with a small numerical aperture, an interferometric measurement structure, a spectrometer, a spectrally broadband illumination and an evaluation unit. Here, the scanning element generates the 2D deflection of the measurement beam required for the optical coherence measurement.

In an advantageous embodiment, the further system is configured to use the near infrared femtosecond laser radiation as illumination radiation. Thus, the illumination when recording the overview image can be implemented, in particular, by the femtosecond laser radiation source.

According to one embodiment, the CCD camera or CMOS camera is arranged laterally at the front of the measuring head in accordance with the Scheimpflug principle.

The arrangement of camera, sensor, imaging lens and object is advantageously implemented according to the “Scheimpflug principle”, in which the object plane, image plane and lens plane have a common axis of intersection. The angles between the planes are designed such that the imaging condition is satisfied at all object points and conjugate image points. The lateral imaging with non-parallel object and image planes results in different imaging scales within the image field. These image distortions are corrected by a computer-based perspective rectification.

As an alternative thereto, camera-based imaging can be provided, wherein the CCD camera or CMOS camera uses the focusing optical unit, in particular the multi-photon focusing optical unit, as an imaging element.

By using the focusing optical unit as imaging constituent part of the camera arrangement, a very compact installation size of the system is advantageously achieved. The camera can advantageously be positioned in the interior of the measuring head. Here, the imaging condition is satisfied by an object distance that is significantly greater than the working distance between the focusing optical unit and the examination object, for example greater by a factor of 10, a factor of 20, a factor of 30 or more than a factor of 50, in particular between a factor of 20 and a factor of 70, e.g., a factor of 50. The optimal object distance is defined by the desired object size, i.e., the edge length of the overview image. As a result of the large object distance and the high numerical aperture of the focusing optical unit, a large-area illumination of the tissue is provided by the excitation beam of linearly polarized femtosecond laser radiation. In the case of a focusing optical unit with a focal length of approximately 4.1 mm, object distances of 2 to 10 mm represent typical object distances. It is known that the focusing optical unit in this arrangement is optically uncorrected and corresponding (mono)chromatic aberrations have to be taken into account. In particular, image distortions are induced, which are corrected by a computer-based perspective rectification.

For the camera sensor, coupling of the illumination beam path and of the imaging beam path can be implemented, for example, by a descanned positioned polarization beam splitter. Unpolarized excitation radiation that was reflected by the tissue and captured by the focusing optical unit is partly reflected by the descanned positioned polarization beam splitter and guided to the camera sensor via a lens system. At this point, chromatic aberrations can be kept low by way of a narrowband illumination. Here, narrowband can denote, for example, a bandwidth of less than 50 nm, preferably less than 20 nm, further preferably less than 10 nm. Alternatively, the polarization beam splitter can be replaced by a beam splitter with splitting by intensity.

Provision can be made for the camera for overview imaging to be introduced manually. To this end, the camera is initially coupled to the adapter plate. After the overview image has been recorded, the camera is removed and the measuring head with the device for high-resolution microscopic imaging is attached.

By way of example, a camera can be provided as a further system, said camera comprising an illumination unit for generating visual or near-infrared light as a further radiation source for the camera-based imaging.

Should this camera be arranged in “non-descanned” fashion, it is expedient if the multi-photon imaging and the imaging by the confocal detection device are unimpaired by the further radiation source since the imaging beam path and the excitation beam path partly intersect. Therefore, for the further radiation source, a further radiation source with a spectral range which does not intersect with the spectral range of the detection radiation or excitation radiation of the multi-photon imaging or of the imaging by the confocal detection device is proposed. By way of example, this condition is satisfied by a spectral range above 950 nm and by the use of a beam splitter in the beam path.

In an advantageous embodiment, for reducing an amplitude of the central reflection, the confocal detection device is arranged in such a way that it taps a partly transmitted signal of a deflection mirror arranged in front of the radiation source. Provision can be made for the signal to be subsequently guided in a second light guide.

Here, the deflection mirror can have a reflectivity of more than 80%, preferably of more than 90%, further preferably of more than 95%. After passing through an analyzer and a lens, the transmitted signal is either directly given to a confocal detector, arranged in the measuring head, with an upstream pinhole or firstly coupled into a light guide and then given to an externally arranged confocal detector. Here, the light guide adopts the function of the pinhole.

Advantageously, the image artifacts in the form of a “central reflection”, which occur in the confocal detection device on account of surface reflections of the illumination radiation at the various optical components, are reduced by way of the crossed polarization detection with the analyzer.

Alternatively, for reducing an amplitude of the central reflection, a polarization beam splitter for separating the linearly polarized excitation radiation and reflected unpolarized secondary radiation of the examination object is provided, wherein the confocal detection device is arranged in such a way that it receives the diffusely reflected unpolarized secondary radiation of the examination object. In this arrangement, the linearly polarized signal of the radiation source will pass through the polarization beam splitter and will be focused in the examination object by means of the focusing optical unit. The radiation is reflected in the focal volume and partly captured in the backward direction by the focusing optical unit after multiple scattering events. The polarization state is disturbed by the multiple scattering event at complex tissue structures in the examination object. The (descanned) signal guided in the backward direction via the scanners is partly output-coupled by the polarization beam splitter and captured in confocal fashion, i.e., after passing through a lens, either given directly to a confocal detector, arranged in the measuring head, with an upstream pinhole or initially coupled into a light guide, the core diameter of which corresponds to the diameter of, e.g., an Airy disk, and then given to an externally arranged confocal detector. As a result of this likewise crossed detection, some of the surface reflections are suppressed and the amplitude of the central reflection is reduced.

In an advantageous embodiment, the confocal detection device comprises an apparatus for time-resolved signal processing of the diffusely reflected signal of the excitation beam of near infrared femtosecond laser radiation.

A further reduction, and ideally a complete compensation, of the central reflection can be achieved by way of the time-resolved detection of the confocally detected signals. Here, either the aforementioned system for time-resolved single photon counting (TCSPC), destined for the fluorescence lifetime measurement, or a simple and cost-effective gated photon counting (GPC) circuit lend itself to this end. With a TTS-limited time resolution of the detectors of, for example, 200 ps, this results in a path resolution of 60 mm in air (TTS, transient time spread). In the case of a time resolution of 20 ps, this path in air is already 6 mm. Here, the surface reflections of most optical units in the excitation beam path can be eliminated.

A further advantageous embodiment lies in the provision of an autofocus unit within the multi-modal imaging system. This unit can be implemented by a measurement system, an evaluation unit and a linear actuator, which may also be referred to as a z-actuator, and can serve for positioning or adjusting the focusing optical unit in relation to the axially moving examination object.

The coupling between the examination object and the imaging system, in particular, can be stabilized by means of the autofocus unit, hence allowing a substantially better examination of living tissue. The fixation of the tissue in the lateral direction is often stabilized very well by the combination of double-sided adhesive tape, a coverslip and a magnetic adapter ring. Then, in the axial direction, the proposed autofocus unit stabilizes the object plane and the focal position, which change, in particular, as a result of pulse, respiration or any other relative movement of the examination object. In particular, the autofocus unit can reduce, or ideally prevent, the image distortion induced by the flexibility of the employed coverslips (by way of example, use is made of coverslips with a thickness of 60 to 180 μm, which bend in the case of an elevated contact pressure of the examination object). Below, image distortions caused in this way are also referred to as “image artifacts”. Respiration artifacts can have a disruptive effect, particularly in the case of highly dynamic tissues, for example when examining skin areas of the chest. Without such an adjustment by an autofocus, the depth information important to the image evaluation is lost, as a result of which vertical slice images, so-called xz- or yz-scans, are distorted and a computer-based evaluation and a 3D reconstruction of the tissue is difficult, particularly in the case of en-face stack recordings.

By way of example, the z-actuator is a stepper motor or can be based on piezo-electronics. Piezo-actuators allow a quick adjustment with a limit frequency greater than 0.1 kHz and are consequently significantly faster than most of the aforementioned relative movements of living objects. Depending on the embodiment, said piezo-actuators offer a travel of 100 μm to 500 μm.

In an advantageous embodiment, for the purposes of providing an autofocus function, the multi-modal imaging system comprises an apparatus for determining the relative position of a coverslip and/or of the examination object.

Advantageously, the sensor signal for the autofocus unit can be provided by a surface reflection of the employed flexible cover slip. Here, the surface reflection can be dynamically evaluated, for example by the integrated system for optical coherence tomography or by way of laser triangulation using a separate system.

According to an advantageous embodiment, the multi-modal imaging system comprises an OC measurement beam aligned collinearly with the optical axis of the focusing optical unit, the position of a coverslip and/or of the examination object being determinable with the aid of said OC measurement beam, for the purposes of providing the autofocus function.

The optical coherence (OC) measurement can advantageously be integrated into the system without imaging as part of the autofocus unit. Here, an OC measurement beam is superposed on the optical axis of the focusing optical unit by way of a beam splitter. To facilitate a low effective numerical aperture and consequently a long Rayleigh length of the OC measurement beam, the numerical aperture of the focusing optical unit is not fully illuminated, but only, for example, to less than 30%, preferably to less than 20%, further preferably to less than 10%. The optical layer of the beam splitter facilitates the transmission of illumination radiation of the multi-photon imaging and of the imaging by the confocal detection device in the spectral range of 710 to 950 nm, for example. The secondary signals to be detected, for example fluorescence and SHG signals in the spectral range of less than 710 nm, and the spectral range for the OC measurement beam are reflected. By way of example, superluminescent diodes (SLD) can be provided for integration, which have, for example, a central wavelength around 1060 nm with a bandwidth of 70 nm, in combination with fiber optic components and integrated spectrometers. An advantage of this arrangement is that the remaining imaging modalities described herein are not impaired. Moreover, the small installation size of this unit and the option of a distal arrangement of the superluminescent diodes and the spectrometers, for example in a mobile trolley housing, have a positive effect on the weight and built volume of the measuring head.

According to an advantageous embodiment, a pressure sensor or a force sensor is provided for finding a surface of the examination object and/or for monitoring a contact pressure.

The axial position of the object depends on the contact pressure. Advantageously, the sensor signal for the autofocus unit can therefore be provided by the pressure sensor. The pressure sensor is preferably an integrated electrical pressure sensor and can be based on strain gauge technology, for example.

Advantageously, the pressure sensor can also simplify finding the tissue surface when initializing the high-resolution imaging. Initializing the image recording can be improved by the use of the linear actuator, which axially displaces the focusing optical unit in relation to the examination object, for example by virtue of there being a continuous axial deflection and the evaluation of the multi-photon fluorescence signals in the ROI being detected at the same time. Here, the surface of the examination object can be defined by the transition from the low-signal coverslip or immersion fluid to the fluorescing sample; cf. FIG. 9 in this respect. By way of example, the turning point from low-signal to high-signal regions is defined as the tissue surface.

The contact pressure can advantageously be controlled or limited by evaluating the pressure sensor. Particularly in the case of ophthalmological and dermatological examinations, this can prevent the occurrence of an excessive contact pressure, which can restrict the blood supply to the tissue and hence impair the metabolism of the tissue.

According to an advantageous embodiment, a release controller for the excitation beam is coupled to an apparatus for determining the presence of the examination object in the measurement region.

By way of example, the apparatus for determining the presence of the examination object can be realized by the above-described pressure sensor. Therefore, the pressure sensor advantageously serves for patient safety. If the pressure sensor registers the examination object, the excitation beam can be released.

As an alternative or in addition thereto, provision can be made for the measurement beam for the optical coherence tomography to serve for determining the presence of the examination object in the measurement region and consequently to be advantageously used for patient safety. By way of example, if tissue is situated in the object plane, it is registered by the measurement beam for the optical coherence measurement and the excitation beam is subsequently released.

By way of example, a computer system of the multi-modal imaging system comprises control and processing units, in particular known signal processing and/or image processing. Moreover, the multi-modal imaging system in particular comprises, for example, user interfaces, such as, e.g., a mouse, a keyboard, displays, a touchscreen or the like. By way of example, the overview image can be displayed by way of a user interface and the user can make a decision about the position of the measurement position to be examined in detail. When recording the detailed image of the examination object, the overview image can be presented in superimposed fashion such that the user recognizes the position in the overview image where the detailed image is recorded. The measurement position can be selected by way of the same user interface (touchscreen) or by way of a further user interface (mouse, keyboard or the like). By way of example, the images can be stored or printed and/or transferred to other modules. Here, the computer system can assist the user, for example by way of suitable zoom and scroll functions. By way of example, further tools can assist the user with the calculation of distances and sizes in the image.

The computer units and peripheral devices of the computer systems, such as screens, input and output devices, etc., can advantageously be situated outside of the measuring head to prevent the latter from becoming unnecessarily heavy.

In a further advantageous embodiment, the multi-modal imaging system is battery-operated.

This embodiment advantageously facilitates a temporary use of the compact system in any surroundings, independently of the presence of a power source. Advantageously, the battery-operated system can be operated independently for a plurality of hours. In particular, this allows the battery-operated system to be moved, facilitating examinations directly at a patient's bed or “in the field”. Moreover, there advantageously is a greater reliability in relation to data losses as a result of a power failure.

In a method according to the invention for non-invasive examination of an examination object using one of the above-described multi-modal imaging systems, the following steps are carried out:
aligning the focusing optical unit with a measurement position,
directing the near infrared femtosecond laser radiation of the radiation source at the measurement position, and
measuring the emitted secondary radiation of the examination object for creating a high-resolution detailed image of the examination object at the measurement position, either successively or simultaneously by the multi-photon imaging system and by the confocal detection device.

Here, the features described above in relation to the system should also be considered to be disclosed for the method, and vice versa.

The step of measuring the emitted secondary radiation of the examination object for creating the high-resolution detailed image of the examination object at the measurement position advantageously comprises a combined measurement by a plurality of systems. Particularly advantageously, the invention provides a combination of multi-photon imaging with high frame rates of imaging by the confocal detection device.

On account of the strong instantaneous reflection signals, significantly shorter pixel dwell times are advantageously required in comparison with autofluorescence-based multi-photon imaging in order to facilitate sufficient morphological imaging of the examination object. This property of the confocal detection device can advantageously be used for “fast” overview imaging of the examination object. Here, overview images can be generated by the checkerboard-like stitching of a multiplicity of adjacent microscopic image fields, for example a multiplicity of adjacent image fields of, e.g., 350×350 μm2. The images are recorded in succession and stitched together in checkerboard-like fashion to form an overview image.

Thus, an overview image can be measured in one embodiment of the method. Here, the method can comprise the following steps:
aligning the measuring head with an overview region of the examination object,
recording an overview image of the examination object by the confocal detection system and/or by a CCD camera or CMOS camera and/or an optical coherence tomography device, and
selecting a measurement position in the overview region for the purposes of recording the high-resolution detailed image.

The illumination when recording the overview image can be implemented, in particular, by the femtosecond laser radiation source.

As described, the high-resolution detailed image can be measured by
aligning the focusing optical unit with the measurement position,
directing the near infrared femtosecond laser radiation of the radiation source at the measurement position, and
measuring the emitted secondary radiation of the examination object for creating a high-resolution detailed image of the examination object at the measurement position by the multi-photon imaging system and/or by the confocal detection device.

In a development of the method, an overview image can initially be implemented by the camera, this can be followed by imaging by the confocal detection device and, finally, by a measurement by way of the high-resolution multi-photon imaging. Here, too, the illumination can always be implemented by the femtosecond laser radiation source.

In one advantageous embodiment, provision is made for axial movement artifacts of the examination object to be corrected by an autofocus function by virtue of the distance between the focusing optical unit and the examination object being continuously mechanically adjusted.

Further, provision can advantageously be made for measurement signals to be evaluated with the aim of finding the surface of the examination object, in particular for providing an autofocus function.

According to an advantageous embodiment, a greater distance between the focusing optical unit and examination object is set when recording the overview image of the examination object than when recording the detailed image of the examination object. In particular, the distance can be set by the linear actuator, which axially displaces the focusing optical unit in relation to the examination object.

As an alternative thereto, the overview image can be recorded as an oblique image by the CCD camera or CMOS camera and/or by the optical coherence tomography device in a manner not collinear with respect to the optical axis of the focusing optical unit.

According to an advantageous embodiment, the excitation beam is released if the examination object is present in the measurement region. By way of example, this can ensure patient safety.

To find the position of the tissue surface in relation to the object plane of the high-resolution imaging system, it is possible in one embodiment to read multi-photon fluorescence signals, which are generated by the creatine in the skin surface. A more precise localization of the skin surface can be ascertained by determining the turning point between low-signal regions of the glass or the water immersion layer and the creatine-rich skin surface.

According to a further aspect of the invention, one of the multi-modal imaging systems described is used for examining living matter of the examination object. Therefore, the method is particularly suitable for the in vivo examination of humans, animals or plants. As a result of the proposed arrangement with a freely positionable flexible measuring head, an examination of object surfaces aligned in any way can be implemented non-invasively, in particular, for example, also by way of a multiplicity of measurements at different parts of an extensive examination object, for example.

In particular, provision can be made for an application on samples which have been marked with biological fluorescence markers, such as GFP, for example.

In general, the distribution of typical endogenous fluorophores such as collagen, elastin, flavins, keratin, NAD(P)H and/or melanin within the tissue can be determined, and, e.g., conclusions can be drawn therefrom about pathological tissue summations, such as cancer, for example.

By doubling the frequency of the excitation radiation, it is however also possible to render collagen fibers or myosin, for example, very clearly visible.

The system is therefore also suitable for the diagnostic support of a physician who, in particular, receives in vivo detailed images of the human skin and can obtain additional support for their diagnostic decisions therefrom.

### BRIEF DESCRIPTION OF THE FIGURES

The figures illustrate possible exemplary embodiments of the invention.

In detail:

FIG. 1 shows a schematic illustration of a multi-modal imaging system as per one embodiment of the invention,

FIG. 2 shows a schematic illustration of a multi-modal imaging system as per a further embodiment of the invention,

FIG. 3 shows two schematic illustrations of an examination object during an examination by a multi-modal imaging system as per one embodiment of the invention at different times,

FIG. 4 shows a schematic illustration of an examination object during an examination by a multi-modal imaging system as per one embodiment of the invention,

FIG. 5 shows a schematic illustration of an examination object during an examination by a multi-modal imaging system as per a further embodiment of the invention,

FIG. 6 shows a schematic illustration of an examination object during an examination by a multi-modal imaging system as per a further embodiment of the invention,

FIG. 7 shows a schematic illustration of part of the measuring head of a multi-modal imaging system as per a further embodiment of the invention,

FIG. 8 shows a perspective illustration of an examination object during an examination by a multi-modal imaging system as per one embodiment of the invention, and

FIG. 9 shows a graph of signal strength over penetration depth.

### EMBODIMENTS OF THE INVENTION

The exemplary embodiments described below with reference to figures should not be construed as restricting the subject matter of the invention. The figures represent the subject matter of the invention only schematically.

In the figures, the same or similar components have been provided with the same reference sign, with repeat mention of these components being dispensed with in the description in individual cases.

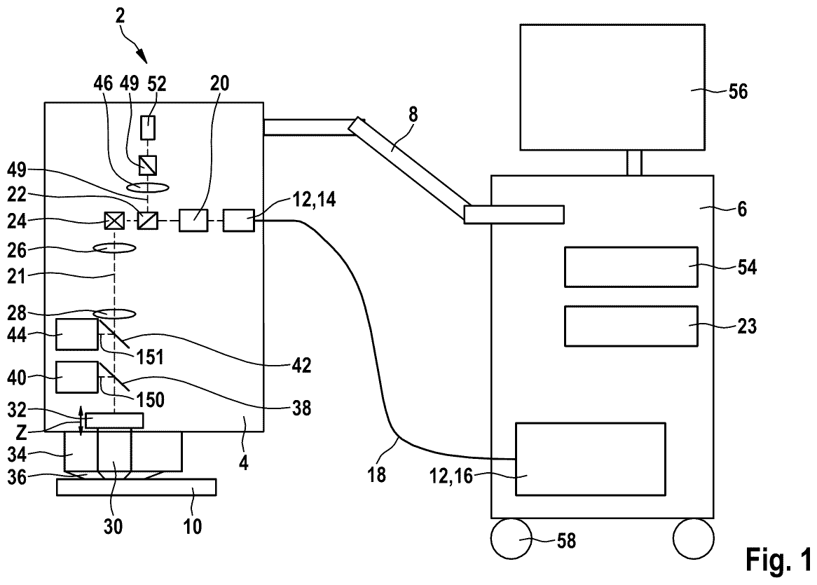

FIG. 1 shows a schematic illustration of a multi-modal imaging system 2 as per a first embodiment of the invention.

The multi-modal imaging system 2 comprises a measuring head 4 which is pivotable, rotatable and flexibly positionable freely in space such that an examination of an examination object 10 is performable under any desired solid angle.

For the purposes of positioning the measuring head 4 freely, the latter is fastened to a mobile base device 6 by means of an articulated arm 8.

The multi-modal imaging system 2 comprises a radiation source 12, which comprises a laser head 14 arranged in the measuring head and a laser driver 16 arranged in the mobile base device 6. The laser head 14 and the laser driver 16 are interconnected by way of a first light guide 18, for example an optical fiber. In some embodiments (not illustrated), the first light guide 18 can be integrated in the articulated arm 8.

By way of example, the laser driver 16 comprises the power supply of the laser and pump diodes. By way of example, the laser head 14 comprises an SHG unit, which doubles the frequency of the radiation emitted by the laser driver 16 for the purposes of generating a linearly polarized excitation beam 21.

The excitation beam 21 passes a power setting unit 20 and a polarization beam splitter 22 and is steered to a focusing optical unit 30 via a scanning unit 24 and via two lenses 26, 28, said focusing optical unit focusing the excitation beam 21 on the examination object 10. The scanning unit 24 allows an angular deflection of the excitation radiation in two planes, which is converted into a two-dimensional translational movement of the excitation volume by means of the focusing optical unit 30.

The focusing optical unit 30 is adjustable in a z-direction, which is indicated by arrows, by means of a linear actuator 32. To this end, the linear actuator 32 comprises a motor. In the illustrated exemplary embodiment, the z-direction corresponds to the optical axis of the focusing unit 30.

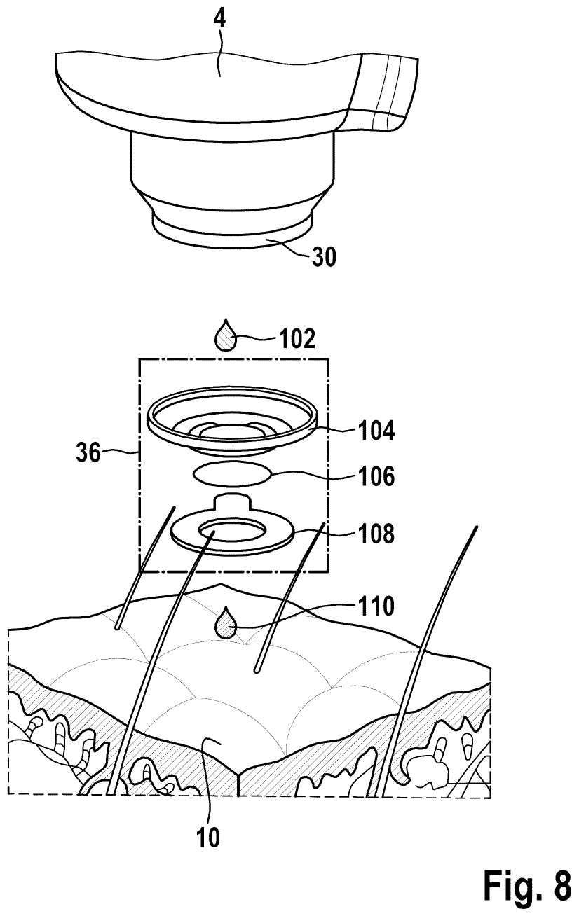

The entire measuring head 4 is locked at any desired position in space by the articulated arm 8. An adapter plate 36, which comes into contact with the examination object 10, is provided between the focusing optical unit 30 and the examination object 10. The adapter plate 36 is described in more detail with reference to FIG. 8. An xy-translation stage 34 is configured to laterally move the adapter plate 36 and the examination object 10 coupled to the adapter plate 36 with respect to the measuring head 4. To this end, the xy-translation stage 34 comprises appropriate motor-driven actuators.

The excitation beam 21 interacts with the examination object 10. Thereupon, the examination object 10 emits secondary radiation, for example reflected radiation, fluorescence radiation, in particular TPF, SHG or THG, which is received by the focusing optical unit 30. In the illustrated exemplary embodiment, two dichroic beam splitters 38, 42 are provided between the second lens 28 and the focusing optical unit 30, said dichroic beam splitters transmitting the TPF, SHG or THG signals to a first and a second detector 40, 44. By way of example, the first detector 40 can be embodied to capture the SHG signals and the second detector 44 can be embodied to capture the TPF signals. In some embodiments (not illustrated), it is possible that only one of the dichroic beam splitters 38, 42 is positioned between the lens 28 and the focusing optical unit 30. In this embodiment, the spectral separation of the signals can be implemented in one of the coupled first and second beam paths 150, 151 using further beam splitters.

In the illustrated exemplary embodiment, a confocal detection device is integrated in the multi-modal imaging system 2. The confocal detection device comprises the polarization beam splitter 22, a lens 46, a pinhole 49, and a confocal radiation detector 52. The confocal detection device receives part of the unpolarized reflection radiation triggered by the excitation beam 21 at the examination object 10. After passing the scanning unit 24, the reflection radiation is tapped in “descanned” fashion to this end. After passing the scanning unit 24, the signal is reflected at the polarization beam splitter 22 and guided via the lens 46 and the pinhole 49 to the confocal radiation detector 52, which is also referred to as a CLSM detector. In the illustrated exemplary embodiment, the confocal radiation detector 52 is situated in the measuring head 4. The radiation source 12 serves as the illumination for the confocal detection device.

A reduction in the amplitude of the central reflection is advantageously achieved by the above-described tapping of the signal whose polarization state differs from that of the excitation beam. In some embodiments, a further reduction or complete compensation can be implemented by way of a time-resolved detection of the confocal signal, with the confocal radiation detector 52 comprising appropriate apparatuses to this end, for example a gate circuit.

In an alternative embodiment, not shown, for capturing the confocally detected fluorescence, the polarization beam splitter 22 can be replaced by a dichroic mirror, the dichroic beam splitters 38, 42 removed, and the detection beam path 160 supplemented by a fluorescence bandpass filter.

In addition to the laser driver 16, the mobile base device 6 typically also comprises signal inputs (not shown) for the signals or images for multi-photon and CLSM imaging, received by the detectors 40, 44 and by the confocal radiation detector 52.

Moreover, the mobile base device 6 comprises a control unit 54, by means of which user inputs, for example, can be processed, the individual components, such as the power sources, can be controlled, and by means of which, also, a change between the overview image and detailed image of the examination object 10, as initiated by a user or by a trigger unit, can be processed. As part of the interface for the user, the mobile base device comprises a display 56, for example a touchscreen or a monitor.

Moreover, wheels 58 are indicated in order to show that the multi-modal imaging system 2 is mobile. A battery unit 23 is provided as a power supply for the multi-modal imaging system 2.

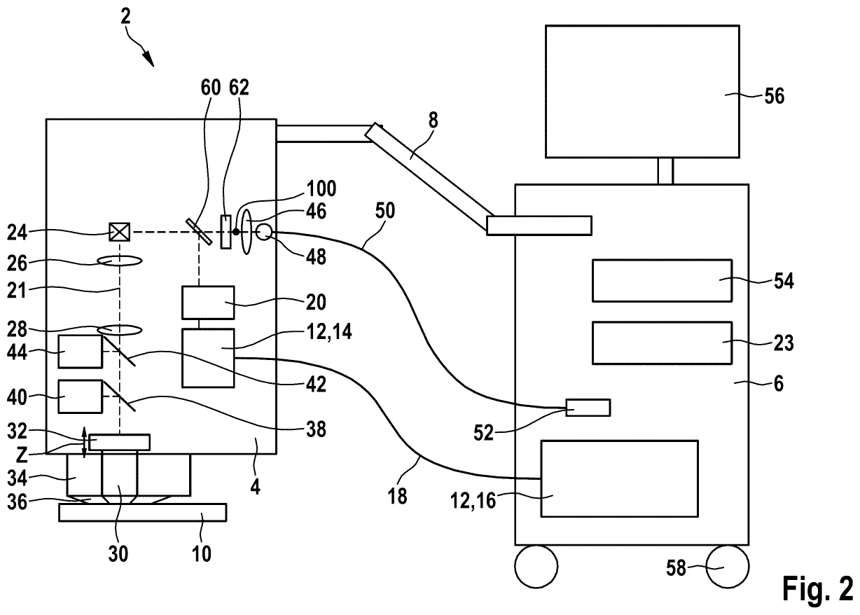

FIG. 2 shows an alternative embodiment of the multi-modal imaging system 2. The multi-modal imaging system 2 illustrated in FIG. 2 likewise comprises a confocal detection device.

The excitation beam 21 generated by the radiation source 12 is initially deflected by a deflection mirror 60 with a high reflectivity, for example more than 90% or more than 95%, but less than 100%, in this embodiment. The further beam path of the excitation beam 21 is as described with reference to FIG. 1. The secondary signal captured by the focusing optical unit 30 at the examination object 10 is guided back by the scanning unit 24 as descanned signal.

The confocal detection device taps the secondary signal as partially transmitted signal of the deflection mirror 60. To this end, after passing the deflection mirror 60, the partially transmitted signal is guided via an analyzer 62, which is aligned such that the polarization states of the excitation radiation 21 and the transmitted detection radiation 100 are polarized orthogonal to one another (crossed polarization). In the exemplary embodiment illustrated here, the transmitted detection radiation 100 is coupled into a second light guide 50 at an input coupling point 48, which is situated in the measuring head 4, via the lens 46, the core diameter of said second light guide corresponding to the diameter of, e.g., an Airy disk, and said transmitted detection radiation is supplied to the confocal radiation detector 52. In the illustrated exemplary embodiment, the confocal radiation detector 52 is situated in the base device 6. This exemplary embodiment with detection in a crossed polarization arrangement also facilitates an effective suppression of the central reflection.

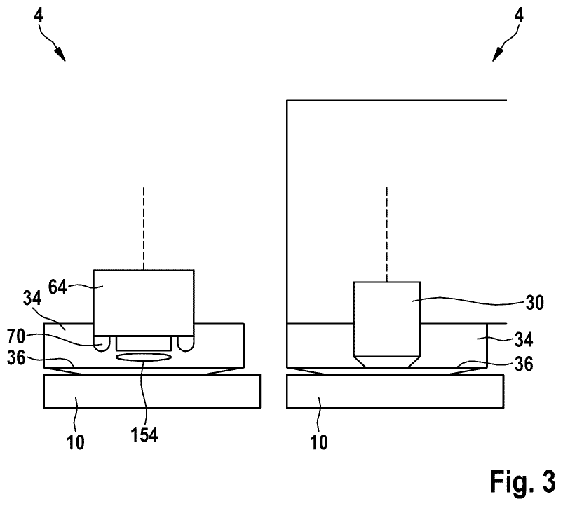

FIG. 3 shows an examination object 10 during an examination by a multi-modal imaging system 2 according to one embodiment of the invention at different times, with part of the measuring head 4 being illustrated in each case, said measuring head facilitating imaging as described with reference to FIG. 1 or 2, for example.

The left half of FIG. 3 illustrates the camera-based overview imaging. To this end, a camera 64 is coupled to the adapter plate 36. In the illustrated exemplary embodiment, the camera 64 comprises a further radiation source 70 for generating visible radiation (VIS), e.g., white light, and a camera lens 154.

Following the overview recording, the camera 64 is removed and the measuring head 4 with the device for high-resolution microscopic imaging is positioned with respect to the examination object 10, as illustrated on the right in FIG. 2.

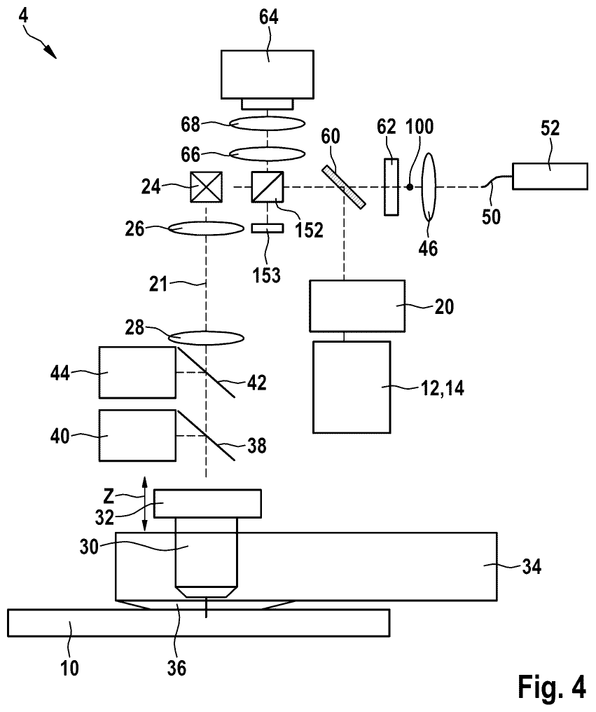

FIG. 4 shows an alternative embodiment of a multi-modal imaging system 2, which couples the multi-photon imaging with the confocal detection device and, additionally, with camera-based imaging.

The beam path of the multi-photon imaging and the confocal detection device can be configured as described with reference to FIG. 2. The camera 64 is completely integrated in the measuring head 4. The camera 64 uses the focusing optical unit 30 as imaging element and the radiation source 12 as illumination. The imaging condition for the camera-based imaging is satisfied by virtue of a greater working distance being set between the focusing optical unit 30 and the examination object 10. By way of example, using the linear actuator 32 to position the focusing optical unit 30 at a distance of approximately 10 mm from the examination object 10 is advantageous here. As a result of the large object distance and the high numerical aperture of the focusing optical unit 30, a large-area illumination of the tissue is provided by the excitation beam 21 of the radiation source 12. The signal passes the scanning unit 24 and is reflected at an intensity beam splitter 152. For this purpose, the intensity beam splitter 152 has an asymmetric reflection/transmission ratio, e.g., less than 20/80, preferably less than 10/90, e.g., 05/95. A beam blocker component 153 is inserted for the purposes of blocking a back side reflection of the intensity beam splitter 152. Then, the signal is fed via a fourth and a fifth lens 66, 68 to the camera 64 and detected there in spatially resolved fashion by the camera sensor. In this configuration, the examination object 10 is situated in the object plane and the camera chip is situated in the image plane of the imaging optical system. The fourth and fifth lens 66 and 68 can also be supplemented by a significantly more complex lens system. It is understood that the measuring head 4 can have appropriate interfaces (not illustrated) for transferring the camera image to the mobile base device 6.

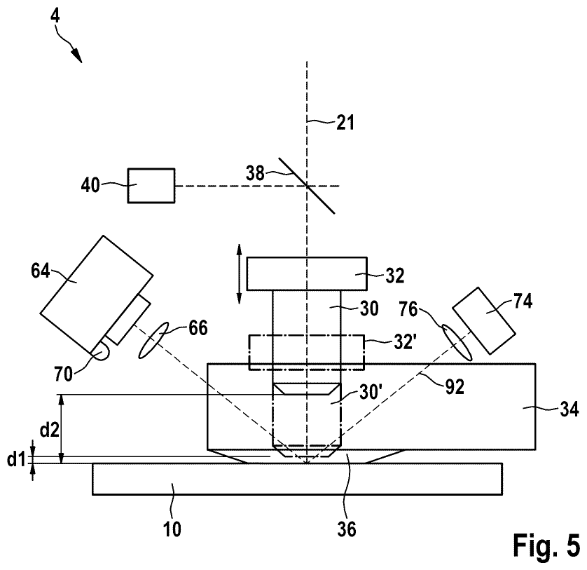

FIG. 5 shows a multi-modal imaging system 2 as per a further embodiment. In addition to the components of the multi-photon imaging and the confocal detection device described with reference to FIG. 1 or 2, for example, the multi-modal imaging system 2 comprises a camera 64 that is integrated laterally on or in the measuring head 4, wherein the arrangement of the camera sensor 64, the imaging lens 66 and the examination object 10 is implemented according to the Scheimpflug principle, in which the object plane, image plane and lens plane have a common axis of intersection. The camera 64 can use the radiation source 12 as illumination. As an alternative or in addition thereto, the further radiation source 70 for generating VIS light is separately arranged on the side as an independent unit, integrated on or in the measuring head.

The focusing optical unit 30 can be adjusted in the z-direction by way of the linear actuator 32, two different settings being illustrated in FIG. 5. At a first distance, the multi-modal imaging system 2 is aligned for recording detailed images of the examination object 10 and has an object distance d1 of, e.g., 0.2 mm (indicated by the reference signs with apostrophes 30′, 32′ for the linear actuator and the focusing optical unit). At a second object distance d2 of, e.g., 10 mm (illustrated by reference signs without apostrophes), the multi-modal imaging system 2 is configured to record the camera-based overview images. In the second case, the lateral and tilted arrangement offers the necessary optical accessibility for the camera-based imaging, without the object-side aperture angle of the camera-based imaging system being clipped by the external dimensions of the focusing optical unit 30.

As a further system, the multi-modal imaging system 2 moreover comprises an optical coherence tomography device, which is indicated in FIG. 5 by an optical coherence measurement system 74, comprising an interferometric measurement structure, spectrometer, illumination, and evaluation unit, and a further focusing optical unit 76. To integrate the optical coherence measurement, superluminescent diodes with a central wavelength around 1060 nm and a bandwidth of 70 nm are used for illumination purposes. The optical coherence measurement system 74, the further focusing optical unit 76 and an OC measurement beam 92 of the optical coherence tomography device are tilted at an angle of, e.g., 30° to 60°, preferably from 40° to 50°, particularly preferably from 42° to 48°, in particular for example 45° in relation to the optical axis of the excitation beam 21. As a result of this, the optical coherence tomography device can make an overview recording of the examination object, for example of the order of 5 mm×5 mm×1 mm when the second distance d2 is set. The lateral and tilted arrangement offers the necessary optical accessibility for the optical coherence tomography device.

There can be a two-dimensional deflection of the OC measurement beam 92 by way of two galvanometer scanners (not shown). Alternatively, there can be a one-dimensional deflection of the measurement beam 92 using only one galvanometer scanner, for example. In the latter case, the 3D imaging can be implemented by synchronizing the resultant line scan with the feed movement of the X- or Y-axis of the motor-driven xy-translation stage 34.

Naturally, there can be embodiments which only comprise the system with the laterally integrated camera 64 or only comprise the optical coherence tomography device that is arranged laterally in tilted fashion.

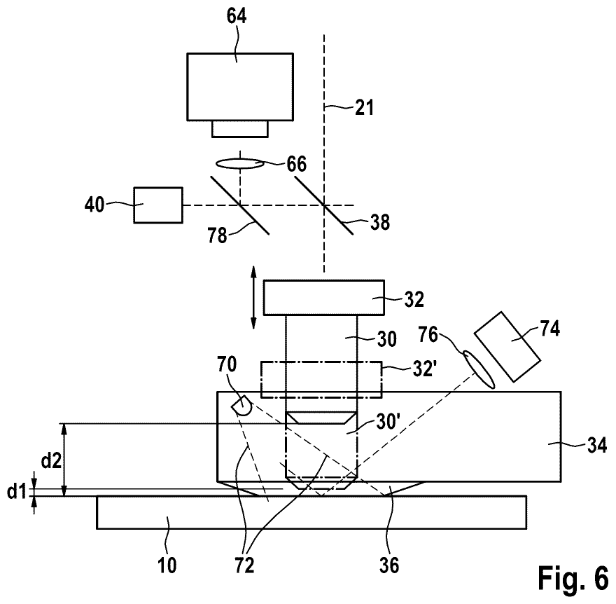

FIG. 6 shows a further configuration of a multi-modal imaging system 2 with multi-photon imaging and an optical coherence tomography device, as described with reference to FIG. 5. As a third system, the camera 64 in this embodiment is not arranged to the side but completely integrated in the measuring head. The camera 64 uses the focusing optical unit 30 as imaging element. In this configuration, the examination object 10 is situated in the object plane and the camera chip in the image plane of the imaging optical system. Reference sign 78 indicates a dichroic beam splitter. A separate further radiation source 70 which is coupled to the focusing unit 30 or to the translation stage 34 (illustrated in FIG. 6) is used for illumination purposes. Here, the spectral range of the excitation radiation 21 and the spectral range of the further radiation source 70 are disjoint. An illuminated region 72 of the further radiation source 70 for overview imaging is also illustrated.

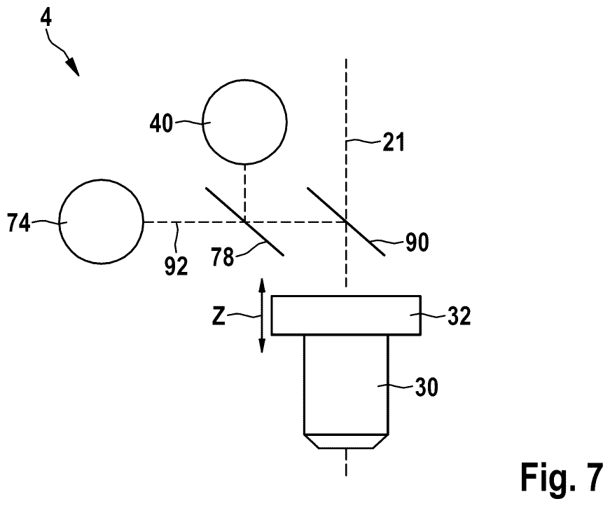

FIG. 7 shows the integration of an autofocus unit by applying optical coherence without imaging. Here, the OC measurement beam 92 from the above-described optical coherence measurement system 74 is coupled into the beam path of the excitation radiation 21 by way of a third dichroic beam splitter 90. Here, the OC measurement beam and the optical axis of the focusing optical unit 30 are aligned in collinear fashion. By way of example, the optical layer of the dichroic beam splitter 90 facilitates the transmission for the illumination radiation with the excitation beam 21 in the spectral range of, e.g., 710 to 950 nm. The detected secondary signals in the spectral range below 710 nm and the spectral range for the OC measurement beam 92 are reflected. In order to obtain only a small effective numerical aperture and consequently a long Rayleigh length of the OC measurement beam 92, the exit pupil of the focusing optical unit 30 is not illuminated completely, but only to about 10% or less.

To integrate the optical coherence measurement, superluminescent diodes with a central wavelength around 1060 nm and a bandwidth of 70 nm are used for illumination purposes. Here, the interferometer is a constituent part of the optical coherence measurement system 74.

FIG. 8 shows a perspective view of a measuring head 4 with an adapter plate 36 and an examination object 10, part of human skin in this case. The adapter plate 36 comprises a metallic coupling ring 104, a coverslip 106, and an adhesive ring 108, in this order. A first immersion liquid, for example an oil, is provided between the focusing optical unit 30 of the measuring head 4 and the metallic coupling ring 104 in order to increase the value of the numerical aperture of the focusing optical unit 30. A second immersion liquid 110, for example water, is provided between the adhesive ring 108 and the examination object 10.

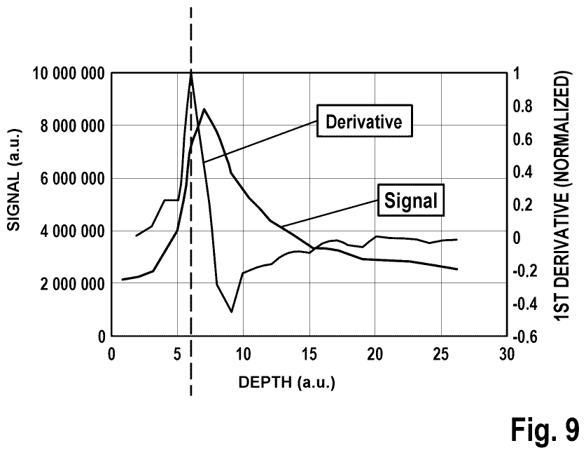

FIG. 9 shows measurement signals for finding the surface of the examination object 10. The surface of the examination object 10 can be defined by the transition from the low-signal coverslip or immersion fluid to the fluorescing sample. By way of example, the turning point from low-signal to high-signal regions is defined as the issue surface.

### LIST OF REFERENCE SIGNS

2 Multi-modal imaging system
4 Measuring head
6 Mobile base device
8 Articulated arm
10 Examination object
12 Radiation source system
14 Laser head
16 Laser driver
18 First light guide
20 Power setting unit
21 Excitation beam
22 Polarization beam splitter
23 Battery unit
24 Scanning unit
26 First lens
28 Second lens
30 Focusing unit
32 Linear actuator
34 xy-translation stage
36 Adapter plate
38 First dichroic beam splitter
40 First detector
42 Second dichroic beam splitter
44 Second detector
46 Third lens
48 Coupling point
49 Pinhole
50 Second light guide
52 Confocal radiation detector
54 Control unit
56 Display
58 Wheel
60 Deflection mirror
62 Analyzer
64 Camera
68 Fifth lens
70 Further radiation source
72 Illuminated region
74 Optical coherence measurement
76 Further focusing optical unit
78 Third dichroic beam splitter
90 Fourth dichroic beam splitter
92 OC measurement beam
100 Transmitted detection radiation
102 First immersion liquid
104 Metallic coupling ring
106 Coverslip
108 Adhesive ring
110 Second immersion liquid
150 First beam path
151 Second beam path
152 Intensity beam splitter
153 Beam blocker component
154 Camera lens
160 Detection beam path
d1 First object distance
d2 Second object distance

What is claimed is:

1. A multi-modal, non-invasive, skin examination and imaging system suitable for examining the skin of a living examination object, comprising:
a multi-photon imaging system operable to provide high-resolution detailed images of the skin, said multi-photon imaging system comprising:
a single radiation source, including a laser head and operable to generate an excitation beam of near infrared femtosecond laser radiation sufficient to trigger emission of secondary radiation by the skin; and
a focusing optical unit, operable to direct the radiation of the radiation source at a measurement position of the skin, wherein the focusing optical unit and the laser head of the radiation source are provided in a measuring head, which is pivotable, rotatable and flexibly positionable freely in space such that the examination of the skin can be performable under any desired solid angle;
at least one confocal detection device, at least partly integrated into the measuring head and configured to receive a reflected signal of the excitation beam of near infrared femtosecond laser radiation reflected by the skin; and
at least one secondary radiation detector at least partly integrated into the measuring head and configured to receive a secondary radiation signal from the skin.

2. The multi-modal imaging system of claim 1, wherein the system is battery operated.

3. The multi-modal imaging system of claim 1, comprising at least one further system for providing overview images of the examination object, wherein the further system is at least partly integrated in the measuring head and comprises a CCD camera or CMOS camera and/or an optical coherence tomography device, wherein the further system is configured to use the near infrared femtosecond laser radiation as illumination radiation.

4. The multi-modal imaging system of claim 3,
wherein the CCD camera or CMOS camera is arranged laterally at the front of the measuring head in accordance with the Scheimpflug principle, or
wherein the CCD camera or CMOS camera uses the focusing optical unit as an imaging element.

5. The multi-modal imaging system of claim 1, wherein the confocal detection device is arranged such that it taps a partly transmitted signal of a deflection mirror arranged in front of the radiation source, in order to reduce an amplitude of a central reflection.

6. The multi-modal imaging system of claim 5, further comprising a polarization beam splitter for separating linearly polarized excitation radiation and diffusely reflected unpolarized secondary radiation of the skin in order to reduce an amplitude of the central reflection.

7. The multi-modal imaging system of claim 5, wherein the confocal detection device is arranged in such that it receives diffusely reflected unpolarized secondary radiation of the skin.

8. The multi-modal imaging system of claim 1, wherein the confocal detection device comprises an apparatus for time-resolved signal processing of a diffusely reflected signal of the excitation beam of near infrared femtosecond laser radiation.

9. The multi-modal imaging system of claim 1, further comprising an apparatus for determining the position of a coverslip and/or of the examination object in order to provide an autofocus function.

10. The multi-modal imaging system of claim 9, further comprising an OC measurement beam aligned collinearly with the optical axis of the focusing optical unit, the position of a coverslip and/or of the examination object being determinable with the aid of said OC measurement beam, for the purposes of providing the autofocus function.

11. The multi-modal imaging system of claim 1, further comprising a pressure sensor for finding a surface of the examination object and/or for monitoring a contact pressure.

12. The multi-modal imaging system of claim 1, further comprising an apparatus for determining a presence of the examination object in the measurement region, said apparatus having a release controller for the excitation beam coupled thereto.

13. The multi-modal imaging system of claim 1, further comprising a scanning unit in the measuring head and operable to steer the excitation beam to the focusing optical unit by angular deflection of the excitation beam in two planes, wherein the scanning unit is positioned to descan the reflected signal before the reflected signal is received by the at least one confocal detection device, and wherein the secondary radiation signal is not descanned by the scanning unit before being received by the at least one secondary radiation detector.

14. A method for non-invasively examining the skin of a living examination object using a system as recited in claim 1, said method comprising:
aligning the focusing optical unit with a measurement position, directing the near infrared femtosecond laser radiation of the radiation source at the measurement position, and
measuring the emitted secondary radiation of the examination object in order to create a high-resolution detailed image of the skin at the measurement position, either successively or simultaneously by the system.

15. The method as claimed in claim 14, further comprising:
aligning the measuring head with an overview region of the skin,
recording an overview image of the skin by the confocal detection system and/or by a CCD camera or CMOS camera and/or an optical coherence tomography device, and
selecting a measurement position in the overview region for the purposes of recording the high-resolution detailed image.

16. The method of claim 14, wherein axial movement artifacts of the skin are corrected by an autofocus function by virtue of the distance between the focusing optical unit and the skin being continuously mechanically adjusted.

17. The method of claim 14, wherein measurement signals are evaluated in order to find the surface of the skin.

18. The method of claim 17, wherein measurement signals are evaluated to provide an autofocus function.

19. The method of claim 14, wherein an overview image is recorded by a CCD camera or CMOS camera and/or by an optical coherence tomography device and
wherein a greater distance between the focusing optical unit and the skin is set when recording the overview image of the skin than when recording the detailed image of the skin and/or
wherein the overview image is recorded as an oblique image by the CCD camera or CMOS camera and/or by the optical coherence tomography device in a manner not collinear with respect to the optical axis of the focusing optical unit.
